(19) United States
(12) Patent Application Publication
(10) Pub. No.: US 2023/0392186 A1
(43) Pub. Date: Dec. 7, 2023

(54) METHOD AND DEVICE FOR DETECTING TRANSGLUTAMINASES

(71) Applicant: TECHNISCHE UNIVERSITÄT DARMSTADT, Darmstadt (DE)

(72) Inventors: Joachim HOENES, Zwingenberg (DE); Katja SCHMITZ, Gross-Zimmern (DE); Natascha KRESTAN, Wiesbaden (DE); Tobias MECKEL, Darmstadt (DE); Christian WEBER, Hamburg (DE)

(21) Appl. No.: 18/326,099

(22) Filed: May 31, 2023

(30) Foreign Application Priority Data
Jun. 1, 2022 (EP) 22176713.0

(51) Int. Cl. C12Q 1/56; B01L 3/00; C12Q 1/48

(52) U.S. Cl. CPC C12Q 1/56; B01L 3/5023; C12Q 1/48; B01L 2300/069; B01L 2300/0825

(57) ABSTRACT

A method and a device for the detection of transglutaminases, in particularly of blood coagulation factor XIII (FXIII). The method is based on a rapid, point-of-care compatible test principle for transglutaminases and allows an exact determination of FXIII in a short time and with simple handling.

### FIELD OF THE INVENTION

The present invention relates to a method and a device for the detection of transglutaminases, in particularly of blood coagulation factor XIII (FXIII). The invention is based on a rapid, point-of-care compatible test principle for transglutaminases and allows an exact determination in a short time and with simple handling.

### BACKGROUND OF THE INVENTION

Blood in the body serves as transport and communication system and is essential for maintaining body functions. When avascular injury occurs, then a rapid wound closure is necessary to keep the blood losses as low as possible. It is achieved by the optimal interaction between thrombocytes and coagulation factors. The main substance of the thrombus is formed by fibrin monomers congregated into polymers. These fibrin monomers are formed by the elimination of low molecular weight peptides from fibrinogen by the serine protease thrombin. For the further stabilization of the formed fibrin network, blood coagulation factor XIII, also called fibrin stabilizing factor, catalyzes a covalent cross-linking of the fibrin molecules among each other. Due to this further stabilization, the thrombus is protected from premature lysis. The mentioned processes represent the final steps of the complex blood coagulation cascade.

Blood coagulation factor XIII (shortly also referred to as “factor XIII” or “FXIII”) is a plasma protein having a weight of 320 kDa. It is present in blood plasma as an inactive heterotetramer with two A and B subunits each. The heterotetramer represents the proenzyme; under physiological conditions FXIII is activated by thrombin. Here, the N-terminal activation peptides are first cleaved by thrombin (FXIII′) and the dissociation equilibrium is shifted toward the monomers. Low calcium concentrations are then sufficient for the activation (FXIIIₐ). Also a non-physiological activation which is independent of thrombin by high calcium concentrations (>100 mM) can dissociate the complex into active monomers (FXIII⁰). Activated FXIII as a transglutaminase links proteins by forming an isopeptide bond between a γ-glutamine residue and the ε-amino group in an exposed lysine side chain. Ammonia is released during this reaction. In the present disclosure, the common three-letter code is also used to designate amino acids or amino acid residues, thus, for example, “Gln” for glutamine or “Lys” for lysine.

A deficiency of FXIII can lead to severe bleedings, delayed wound healing, but also to increased numbers of abortions in women. However, hereditary FXIII deficiency is mostly already diagnosed during infancy due to umbilical stump bleedings. This hereditary form of FXIII deficiency is very rare. An acquired deficiency is more common and is caused, for example, by an impaired synthesis capacity of the liver, chronic inflammatory bowel diseases, sepsis or consumptive coagulopathies. For the detection of an FXIII deficiency, conventional coagulation tests such as thromboplastin time or activated partial prothrombin time (aPTT) are not suitable. The number of thrombocytes and the fibrinogen concentration also do not provide information about FXIII.

Particularly in cases of severe bleedings, e.g., after accidents or during major surgeries, a deficiency of coagulation factors leads to complications such as, e.g., increased blood loss. The administration of whole blood is nowadays more and more avoided, because, on the one hand, it substitutes losses, but, on the other hand, it also creates new imbalances between the factors and it can cause additional complications. Instead, attempts are made to restore the physiological balance by a targeted administration of individual factors. Corresponding preparations are available for fibrinogen and also for FXIII.

Currently, the activity of FXIII can be determined in the clinical chemistry laboratory, e.g., photometrically. For this purpose, FXIII of the sample is activated by thrombin and it links a glutamine donor substrate with an amine donor, e.g., glycine ethyl ester, with the release of ammonia. The resulting ammonia can be determined in a coupled enzymatic reaction. NADH, α-ketoglutarate and free ammonia are converted into NAD⁺ and glutamate by the glutamate dehydrogenase. The decrease of NADH is measured via the absorbance at 340 nm and it is directly proportional to the FXIII activity.

In an alternative, factor XIII can be detected by antibodies in an enzyme-linked immunosorbent assay (ELISA). Here, factor XIII binds to a specific antibody which is immobilized in the test vessel. After washing out other substances, a second antibody binds to the immobilized factor XIII. An enzyme conjugated to the antibody converts the subsequently added signaling substrate. The substance formed can be quantified photometrically or fluorimetrically, and the FXIII level can be determined from this.

Both tests require a transport of the sample to the clinical chemistry laboratory, a centrifugation for plasma isolation, the conducting of the actual test as well as an evaluation and feedback to the client. Such procedures involving the clinical laboratory are too slow (also in the case of other test methods) in acute cases to assess the need for an administration and to determine the correct dosage of the preparations. Alternative test methods are thromboelastometry (rather qualitative and quite susceptible to faults) as well as enzyme immunoassays (very laborious and expensive, no activity but protein determination).

Rapid point-of-care (POC) tests could be applied on-site (e.g., in the operating room) and allow a correct decision to be made within a few minutes, but however have not been available to date for a variety of reasons.

Blood is a highly complex mixture of cells (main constituent erythrocytes) and constituents being dissolved in the serum, such as proteins and lipids. The strong light absorption in hemoglobin makes photometric or fluorimetric determinations in whole blood difficult, if not impossible. Electrochemical determinations are also strongly disturbed by the cells and by the viscosity of the blood matrix. A (partial) correction is possible with methods such as the impedance spectroscopy, but however this is also associated with a great effort.

A rapid separation of the erythrocytes is possible using mats of glass fibers. However, soluble blood constituents remain in the blood plasma thus obtained. Especially the high concentrations of serum-albumin and fibrinogen have the potential to interfere with tests for FXIII. E.g., the dansylcadaverine test, in which a fluorescently labeled amine is incorporated into a glutamine-containing protein, e.g., dimethylcasein, is known. The blue shift and enhancement of the fluorescence due to the altered chemical environment of the fluorophore can be used as a measure for the activity of FXIII. Serum-albumin in physiological concentrations, however, binds the dansylcadaverine, withdrawing it from the enzymatic reaction and shifting the fluorescence spectrum in advance so that any residual reaction that may still be taking place is no longer visible.

Fibrinogen is a further interfering component: the physiological activation of FXIII requires cleavage by thrombin; in an alternative, high Ca²⁺ concentrations can be used. In both cases the coagulation reaction is triggered; the coagulum hinders the detection reactions by light scattering and high viscosity.

The only known method for the determination of FXIII in whole blood is the thromboelastography (EP 2 474 830 A1). In later stages of the coagulation (thus, after a longer observation time), FXIII provides a solidification of the coagulum, the rate and extent of which depend on the sample's content of FXIII. The method is relatively slow due to the longer observation time. In addition, many other ingredients of the blood (cells, fibrinogen, serum-albumin, agents that thin the blood) and the onset of fibrinolysis affect the results, so that exact FXIII determinations are not possible.

### SUMMARY OF THE INVENTION

As a result of these problems, the prior art does not provide a method that would allow an exact determination of transglutaminases, in particularly of FXIII, in a reasonably short time and with simple handling. Against this background, the object of the present invention is to provide a rapid, POC-compatible test principle from human blood.

### BRIEF DESCRIPTION OF THE DRAWINGS

FIG. 1A shows a schematic structure of a device according to the present invention in the form of a lateral flow test. The device contains an application zone 11, a reaction zone 12 and a suction zone 13. The application zone 11, the reaction zone 12 and the suction zone 13 are positioned on a carrier 14. The thickness of the suction zone 13 is higher than the thickness of the application zone 11 and the reaction zone 12 which have the same thickness. For example, it is possible to use paper as a material for the application zone 11, the reaction zone 12 and the suction zone 13 respectively. The three papers can be placed on the carrier 14 (for example a microscope slide, preferably made of plexiglass) with a slight overlap. In contrast to the schematic drawing, in practice the papers usually flexibly lay against and follow the surface of the carrier 14. In an alternative, the zones 11, 12 and 13 can also be realized with a continuous fiber mat, e.g., a paper. This structure is shown in FIG. 1B. A carrier is an optional feature, and it is not shown in FIG. 1B.

FIG. 2 shows the dependence of the signal on the amount of FXIII. With a device such as shown in FIG. 1A, the amine donor dansylcadaverine has been bound to an immobilized FXIII substrate by the transglutaminase FXIII. The bond of dansylcadaverine to the immobilized FXIII substrate results in a change, spectral change of the fluorescence of the dansylcadaverine which has been recorded by means of an imager. The increase of the fluorescence was measured at an excitation wavelength of 365 nm and an emission wavelength of 505-565 nm. In FIG. 2, on the x-axis the amount of the transglutaminase (in International Units [I.U.]) is plotted. On the y-axis the signal as the intensity change ΔI per minute is plotted. The intensity change is the difference of the fluorescence intensity 30 minutes after the addition of the amine donor and the fluorescence intensity 5 minutes after the addition of the amine donor. The change of the fluorescence intensity obtained has been divided by the time period of 25 minutes for obtaining the signal as the intensity change ΔI per minute. A linear relationship between the amount of the transglutaminase (in International Units [I.U.]) and the signal (intensity change ΔI per minute) results.

FIG. 3 shows a bar diagram of the signal (intensity change ΔI per minute)±standard deviation (n=3 independent experiments). The influence of potential interfering components in comparison to a control without these interfering components was investigated. As interfering components serum-albumin, fibrinogen as well as a combination of serum-albumin and fibrinogen were tested. A significant change of the signal through the potential interfering components has not been detected. Thus, the measurement is not influenced by the presence of the respective components.

FIG. 4 shows the dependence of the signal on the amount of FXIII. With a device such as shown in FIG. 1A, the amine donor Lucifer Yellow-cadaverine or the amine donor Lucifer Yellow-CH were bound to an immobilized FXIII substrate by the transglutaminase FXIII. Lucifer Yellow-CH comprises a carbohydrazide chain instead of the cadaverine group. The increase of the fluorescence was measured at an excitation wavelength of 460 nm±20 nm and an emission wavelength of 505-565 nm. In FIG. 4, on the x-axis the amount of the transglutaminase (in International Units [I.U.]) is plotted. On the y-axis the intensity is plotted. A clear relationship between the amount of the transglutaminase (in International Units [I.U.]) and the fluorescence intensity measured results.

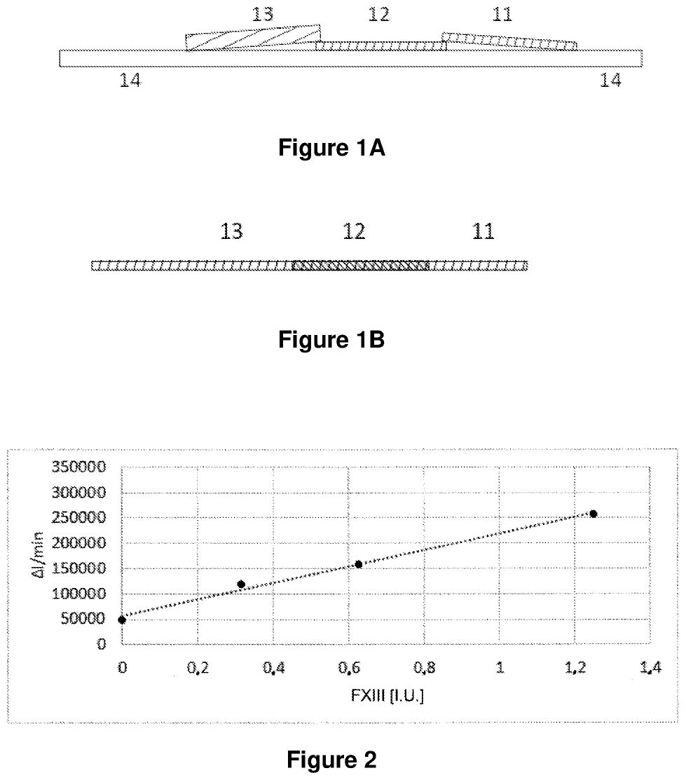

### DETAILED DESCRIPTION OF THE INVENTION

The method of the present invention is suitable for all transglutaminases. The determination of the enzymatic activity of blood coagulation factor XIII (FXIII) in a sample is particularly preferred.

The bond takes place to an immobilized glutamine donor. This donor, at the same time, can be the substrate which is used for the detection, but this is not necessary. For the detection of the activity, additional substrate can be added, either the same glutamine donor or another one.

The detection can be realized either by washing out of unbound amine donor and detecting the one remaining in the reactive field or, preferably, by a spectral shift of the chromophore on the amine donor by the bond. For this purpose, a series of fluorophore groups can be used that respond directly to the altered polarity of the environment, e.g., dansyl, dapoxyl, 4-chloro-7-nitrobenzofurazan (NBD), 6-dimethylamino-2,3-naphthalimide. Such fluorophores can be summarized under the term “spectroscopic polarity sensors” In particularly, the “amount” of incorporated amine can be determined indirectly by spectroscopy; in particularly, the activity of the transglutaminase can be determined by the temporal change of the spectroscopic signal.

As a linker between chromophore and glutamine, in particularly, diamines, preferably putrescine, cadaverine and/or carbohydrazide are suitable.

In a first aspect, the invention relates to a method for the determination of the enzymatic activity of a transglutaminase, in particularly of blood coagulation factor XIII (FXIII), in a sample, wherein the method comprises the following steps:
a) providing of an immobilized glutamine donor, e.g., an FXIII substrate, wherein the glutamine donor is a peptide, in particularly a polypeptide or protein, and wherein the glutamine donor comprises at least one glutamine residue,
b) activation of the transglutaminase which is present in the sample,
c) contacting of the sample with the glutamine donor so that transglutaminase which is present in the sample binds to the glutamine donor,
d) removal of constituents of the sample which are not bound to the glutamine donor,
e) contacting of the transglutaminase which is bound to the glutamine donor with an amine donor so that the amine donor is bound to a glutamine donor substrate by the transglutaminase, and
f) determination of the enzymatic activity of the transglutaminase with the help of a signal which is created by the bond of the amine donor to the glutamine donor substrate.

Preferably, the signal is proportional to the rate of the bond of the amine donor to the glutamine donor substrate which is catalyzed by the transglutaminase.

In particularly, the steps are conducted in the given order a) to f).

The immobilized glutamine donor is preferably the glutamine donor substrate to which the amine donor is bound by the glutaminase.

The transglutaminase is preferably selected from the group of the human transglutaminases. It is particularly preferred, when the transglutaminase is blood coagulation factor XIII (FXIII).

Preferably, the sample contains exactly one transglutaminase.

Preferably, the sample is a blood sample, in particularly a human blood sample. In other embodiments, the sample may also be a tissue sample, for example an extract from a tissue.

The sample contains transglutaminase, in particularly FXIII, preferably in an activity of 5% to 200%, for example of 25% to 175%, of 50% to 150%, of 75% to 125%, or of 90% to 110%, based on the activity in standard plasma. Preferably, the activity is at least 5%, at least 25%, at least 50%, at least 75%, or at least 90%, based on the activity in standard plasma. Preferably, the activity is at most 200%, at most 175%, at most 150%, at most 125%, or at most 110%, based on the activity in standard plasma.

According to step a) of the method according to the present invention, an immobilized glutamine donor, in particularly an immobilized glutamine donor substrate, preferably an immobilized FXIII substrate, is provided. The (FXIII) substrate may be a physiological (FXIII) substrate. In particularly preferred embodiments, the (FXIII) substrate is not a physiological (FXIII) substrate. (FXIII) substrate in particularly means that the transglutaminase (in particularly FXIII) is able to catalyze the reaction of a glutamine residue of the substrate with the amino group of an amine donor. When the glutamine donor indeed comprises at least one glutamine residue, but the transglutaminase (in particularly FXIII) is not able to catalyze a bond between glutamine residue and amine donor, then the glutamine donor is not an (FXIII) substrate.

The glutamine donor may in particularly be a peptide, preferably a polypeptide or protein. The glutamine donor contains at least one glutamine residue. Preferably, the glutamine donor contains several glutamine residues, for example at least two, at least three, or at least five glutamine residues. Preferred are proteins having a molecular weight of 10 to 300 kDa, for example of 10 to 100 kDa or of 12 to 30 kDa. The molecular weight of the glutamine donor may, for example, be at least 10 kDa, or at least 12 kDa. The molecular weight of the glutamine donor may, for example, be at most 300 kDa, at most 100 kDa or at most 30 kDa. Preferably, the content of glutamine of the glutamine donor is in a range of 3% to 15%, based on the total number of residues, for example in a range of 5% to 12% or of 7% to 10%. The content of glutamine of the glutamine donor may, for example, be at least 3%, at least 5% or at least 7%. The content of glutamine of the glutamine donor may, for example, be at most 15%, at most 12% or at most 10%.

It is particularly preferred, when the glutamine donor is a substrate of the transglutaminase, also called glutamine donor substrate. This results in the advantage that the immobilized glutamine donor can function as glutamine donor substrate to which the amine donor is bound by the transglutaminase. This eliminates the need for the addition of additional substrate of the transglutaminase. However, also embodiments are comprised in which the immobilized glutamine donor is not identical with the glutamine donor substrate of step e) of the method of the present invention. In particularly in the case of such embodiments, in addition to the immobilized glutamine donor additional glutamine donor substrate is added to which the amine donor can be bound by the transglutaminase. For example, it is possible, before conducting step e), to provide a mixture or solution which comprises amine donor and glutamine donor substrate, and to contact it in step e) with the transglutaminase which is bound to the glutamine donor so that the bond between amine donor and glutamine donor substrate can be formed by the transglutaminase. The addition of glutamine donor substrate can also be carried out in a case, when the immobilized glutamine donor itself is already a glutamine donor substrate.

In the present disclosure, the terms “peptide” or “peptides” collectively describe molecules containing amino acids which are linked to each other via peptide bonds. This includes oligopeptides (peptides with up to ten amino acid residues) and polypeptides (peptides with more than ten amino acid residues). Polypeptides with more than 50 amino acid residues are also called proteins. The terms “peptide” or “peptides” as used in the present disclosure expressly also comprise proteins.

According to step a) of the method, the glutamine donor, in particularly the FXIII substrate, is present in an immobilized form. The immobilization in particularly comprises the bond of the glutamine donor to a carrier, preferably Sepharose, cellulose or paper, in each case preferably functionalized with DEAE (diethylaminoethyl group) or other cationic groups. In addition or in an alternative, the immobilization may also comprise a precipitation of the glutamine donor, in particularly of the FXIII substrate in the presence of the carrier, for example a precipitation with Ca²⁺.

The immobilized glutamine donor, in particularly the substrate-carrier complex, may be present in different forms. For example, it is possible that the immobilized glutamine donor is present in the form of a suspension, in particularly in a reaction vessel. It is also possible to immobilize the glutamine donor on a solid-state body, for example on a paper carrier in a test strip.

The immobilization of the glutamine donor preferably comprises one or several of the following measures:
precipitation and adsorption
ionic bond to DEAE-functionalized surfaces
covalent bond by formaldehyde, glutardialdehyde
covalent bond using EDC/NHS in the case of amine- or COOH-functionalized carrier surface
covalent bond by dimethyl suberimidate, dimethyl pimelimidate, dimethyl adipimidate in the case of amine-functionalized carrier surface
covalent bond by carrier-bound epoxides, isocyanates
covalent bond via amino groups to BrCN-activated polysaccharides.

The immobilized glutamine donor may comprise modifications, for example chemical modifications. Particularly preferred are modifications of lysine residues. In particularly, the glutamine donor may be methylated, acetylated or succinylated, for example dimethylated, in particularly N,N-dimethylated or also acetamidinated.

In particularly it may be that the glutamine donor is methylated, for example dimethylated, in particularly N,N-dimethylated. The dimethylation is in particularly realized by reaction of the glutamine donor with formaldehyde and sodium borohydride. In an alternative or in addition, one or several other reducing agents can be used. The reaction depends on the accessibility of the lysins—readily accessible reacts fastest. The degree of dimethylation is controlled by the amount of the reagents added and the reaction time. A degree of dimethylation of the freely accessible lysins which is as high as possible is preferred, because this prevents the cross-linking within a protein or between the proteins. Preferably, the degree of dimethylation is higher than 90%.

In addition or in an alternative to the methylation, the immobilized glutamine donor may preferably be succinylated or acetamidinated.

It is particularly preferred, when the glutamine donor is selected from the group consisting of casein, in particularly dimethylcasein, or lysozyme, in particularly succinylated lysozyme, preferably succinylated chicken egg white lysozyme (Gallus gallus). Casein from cow milk (Bos taurus) is preferred. Dimethylcasein is well known to those skilled in the art, as it is commonly used for the determination of protease via the detection of newly formed terminal amino groups.

According to step b) of the method according to the present invention, the transglutaminase which is present in the sample is activated. In embodiments in which the transglutaminase is FXIII, the activation can be realized by thrombin. But in this case, however, the coagulation should be prevented, for example by the addition of inhibitors of the fibrin aggregation, e.g., by the tetrapeptide GPRP.

Preferably, high Ca²⁺ concentrations are used for the activation. This in particularly is true, when the transglutaminase is FXIII, but also in the case of other transglutaminases. When an activation is realized with Ca²⁺, then the Ca²⁺ concentration is preferably in a range of 100 to 500 mM, for example of 200 to 400 mM. The Ca²⁺ concentration may, for example, be at least 100 mM or at least 200 mM. The Ca²⁺ concentration may, for example, be at most 500 mM or at most 400 mM. Also lower Ca²⁺ concentrations, for example concentrations of 1 to <100 mM, in particularly of 1 to 10 mM, in connection with a high ionic strength are possible. A high ionic strength can, for example, be obtained by a NaCl concentration in a range of 200 to 1000 mM, in particularly of 400 to 700 mM.

In embodiments in which the transglutaminase is FXIII, FXIII after the activation according to step b) preferably is present as FXIIIₐ (cleaved by thrombin) or as FXIII⁰ (activated by Ca²⁺ ions).

According to step c) of the method according to the present invention, the sample is brought into contact with the immobilized glutamine donor. The particular details of the contacting basically depend on the kind of the immobilization of the glutamine donor. When the immobilized glutamine donor is present as a suspension, in particularly in a reaction vessel, then the contacting may, for example, be realized by the addition of the sample to the suspension, for example by the addition of the sample into the reaction vessel. But, on the other hand, when the glutamine donor is present in an immobilized form on a solid-state body, such as for example a test strip, the contacting can be realized by bringing the sample and the solid-state body, in particularly the test strip, into contact. For example, the sample can be applied at the site on the solid-state body on which also the glutamine donor is immobilized. This may be a preferred option in particularly in the case, when the solid-state body, in particularly the test strip, is designed such that the sample can migrate through the solid-state body, for example by capillary forces, and thus can be brought into contact with the immobilized glutamine donor.

It is possible to subject the sample to a pretreatment step, for example a purification step, prior to contacting with the immobilized glutamine donor. Preferably, however, the method does not include a step of pretreating the sample. It is a particular advantage of the present invention that the sample does not require any pretreatment. So, in particularly, it is possible to contact the sample, for example a (human) blood sample, with the immobilized glutamine donor after its collection without pretreatment. Calcium can be added. In the case of a precipitation of the glutamine donor with Ca²⁺, the calcium in the precipitated glutamine donor may also contribute to the activation of the transglutaminase or cause the activation.

The contacting according to step c) of the method may comprise an incubation step. The incubation step is particularly advantageous for further increasing the extent of the bond of the transglutaminase to the glutamine donor. Preferably, the incubation time is in a range of 10 seconds to 5 minutes, for example of 0.5 to 5 minutes or of 1 to 3 minutes. The incubation time may, for example, be at least 10 seconds, at least 30 seconds or at least 60 seconds. The incubation time may, for example, be at most 300 seconds or at most 180 seconds. The incubation time is in particularly the time period from the beginning of the contacting according to step c) to the beginning of the removing of constituents of the sample which are not bound to the glutamine donor according to step d).

The contacting of the sample with the immobilized glutamine donor according to step c) of the method in particularly leads to a bond of the transglutaminase to the immobilized glutamine donor.

Glutamine residues are necessary for the bond of the transglutaminase to the immobilized glutamine donor. Transglutaminases may in particularly bind with an essential SH group to a glutamine of the glutamine donor, wherein under elimination of ammonia a thioester is formed, such as is shown in following reaction scheme 1.

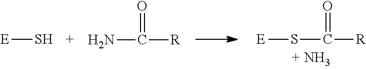

E-SH describes the enzyme (the transglutaminase) with its essential SH group in the active center. From the glutamine donor the side chain of a glutamine residue is shown. The remaining glutamine donor is illustrated as “R”. Under elimination of ammonia a thioester bond is formed, which links the enzyme E with the glutamine donor R.

This reaction also occurs physiologically. However, transglutaminases do not only bind to physiological glutamine donors, but also to other glutamine donors, in particularly such ones that comprise at least one glutamine residue.

The transglutaminase (in particularly FXIII) binds to the immobilized glutamine donor, in particularly to the immobilized glutamine donor substrate. The bond between transglutaminase and glutamine donor is preferably a covalent bond between an amino acid in the active center of the glutaminase and a glutamine residue of the glutamine donor. The bond between transglutaminase and immobilized glutamine donor is in particularly a thioester bond. The thioester bond is preferably formed by reaction of an SH group of the transglutaminase (in particularly the essential SH group) and a glutamine residue of the immobilized glutamine donor.

According to step d) of the method according to the present invention, constituents of the sample which are not bound to the immobilized glutamine donor are removed. One constituent of the sample which binds to the glutamine donor is in particularly the transglutaminase, preferably FXIII. Other constituents of the sample, in particularly constituents which potentially interfere with the detection of the transglutaminase, preferably do not or only weakly bind to the glutamine donor. A slight bond is in particularly a bond which allows the removal with one or several washing steps.

Step d) of removing constituents of the sample which are not bound to the glutamine donor preferably comprises one or several washing steps. The number of the washing steps is preferably in a range of 1 to 3. Particularly preferred is the continuous washing, in particularly by overflowing the carrier of the immobilized glutamine donor. Here, volumes of 1 to 3 times the carrier volume are used. The carrier volume is the volume of the liquid which is present in the carrier.

The particular details of the removal of constituents of the sample which are not bound to the glutamine donor basically depend on the kind of the immobilization of the glutamine donor.

When the immobilized glutamine donor is present as a suspension, in particularly in a reaction vessel, then the removal, for example, may be realized by conducting one or several washing steps, wherein one washing step comprises the separation of the suspension into solid and liquid constituents, the removal of the liquid constituents and the addition of washing solution, in particularly washing buffer. The separation of the suspension into solid and liquid constituents may in particularly be realized by means of centrifugation. Preferred is a centrifugation for 0.5 to 2 minutes, wherein the required acceleration depends on the kind of the carrier.

When, on the other hand, the substrate is present on a solid-state body, such as for example the paper in a test strip, then the removal of constituents of the sample which are not bound to the glutamine donor may, for example, be realized by conducting one or several washing steps, wherein one washing step comprises the addition of washing solution to the solid-state body, in particularly to the test strip. The washing solution is preferably a washing buffer.

The washing solution may, for example, be applied at the site on the solid-state body on which also the glutamine donor is immobilized. However, it is also possible to apply the washing solution at a site on which the glutamine donor is not immobilized. This may be a preferred option in particularly in the case, when the solid-state body, in particularly the test strip, is designed such that the washing solution can migrate through the solid-state body, for example by capillary forces, and thus can be brought into contact with the immobilized glutamine donor. In this way, it can be achieved that constituents of the sample which are not bound to the glutamine donor are removed.

The washing solution or the washing buffer preferably has an approximately isotonic composition, e.g., 100 mM NaCl and 50 mM HEPES. The pH value is preferably in a range of 6.0 to 9.0, further preferably of 7.0 to 8.0. Preferably, the pH value is at least 6.0 or at least 7.0. Preferably, the pH value is at most 9.0 or at most 8.0. The washing solution or the washing buffer may, for example, also be PBS (phosphate-buffered saline), in particularly 137 mM NaCl, 2.7 mM KCl and 12 mM phosphate (HPO₄²⁻ and H₂PO₄⁻), pH value 7.4. The washing solution or the washing buffer may contain one or several biocides, for example NaN₃, preferably in concentrations of 0.01 to 1.0 mM or of 0.05 to 0.5 mM, for example about 0.1 mM.

Physiologically, the thioester between transglutaminase and glutamine donor is attacked by the ε-amino group of a lysine, and under aminolysis the isopeptide bond Gln-Lys is created. The essential SH group is released again. Instead of lysine, the enzyme accepts a whole range of amine donors; also alcohols are incorporated, however less preferred. In the absence of such substrates hydrolysis takes place. Aminolysis and hydrolysis are schematically shown in the following reaction scheme 2.

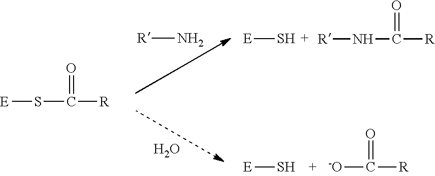

Here, R′—NH₂ designates an amine donor. The hydrolysis proceeds more slowly than the aminolysis, and it is therefore shown with the help of a dashed line.

The aminolysis, such as described in detail below, can be used for the detection of transglutaminase. However, a premature hydrolysis can lead to an early release of the transglutaminase from the immobilized glutamine donor so that the corresponding transglutaminase cannot longer be detected by the aminolysis. Thus, for a sensitivity of the detection of the transglutaminase which is as high as possible, the rate of the hydrolysis should be as low as possible.

For obtaining a bond of the transglutaminase to the immobilized glutamine donor which is as stable as possible it is advantageous, when the glutamine donor comprises more than one glutamine residue. With this, in particularly, it can be achieved that the transglutaminase also by several washing steps cannot or not appreciably be removed from the glutamine donor. So, several washing steps can be conducted and an improved removal of constituents of the sample which are not bound to the glutamine donor can be achieved, without compromising the detection of the transglutaminase by an excessive washing out thereby. Such a strong bond of the transglutaminase to a glutamine donor, in particularly to a glutamine donor substrate is surprising. Without wishing to be bound to any particular theory, the influence of the number of the glutamine residues onto the bond could be explained, at least in part, as follows.

In the case of a glutamine donor comprising diverse glutamines the environment of the glutamine is also a factor which has an influence onto the rates of aminolysis and hydrolysis. In such a system the transglutaminase will bind to one of the glutamines. When the hydrolysis is quick enough, then the enzyme is released again and it can repeat the reaction. On a glutamine with slow hydrolysis, it remains in an immobilized state. The system is self-optimizing. The transglutaminase binds to the glutamine residue with the slowest hydrolysis, either directly or after some attempts on other glutamine residues which are not successful due to hydrolysis. This is shown in the following reaction scheme 3.

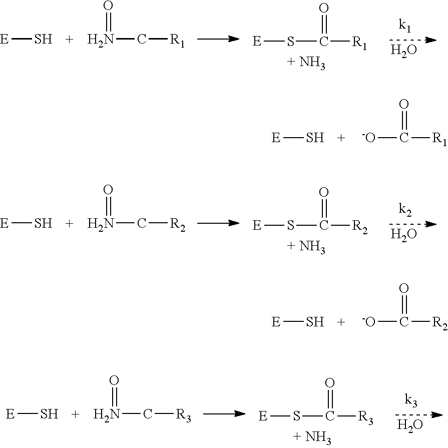

With R₁, R₂ and R₃ it is designated that different glutamine residues of the glutamine donor are meant. k₁, k₂ and k₃ are the different hydrolysis rate constants. The hydrolysis rate of the third glutamine is particularly low and, therefore, it is shown with the help of a dashed line.

Preferably, the glutamine residues are present in a molar excess with respect to the transglutaminase.

According to step e) of the method according to the present invention, the transglutaminase which is bound to the glutamine donor is brought into contact with the amine donor so that the amine donor is bound to a glutamine donor substrate by the transglutaminase. Here, in particularly, the bond between the transglutaminase and the immobilized glutamine donor is broken. When the immobilized glutamine donor itself is a glutamine donor substrate, then the transglutaminase can catalyze the formation of a bond between amine donor and immobilized glutamine donor substrate. When the immobilized glutamine donor is not a glutamine donor substrate, then the bond of the amine donor is not realized to the immobilized glutamine donor, but to the glutamine donor substrate which is present in addition. In particularly in the case of such embodiments, the glutamine donor substrate can, for example, be added separately or together with the amine donor.

The amine donor may contain a chromophore. Preferred, in particularly, is a fluorescent amine donor, particularly preferably dansylcadaverine, dapoxylcadaverine or NBD-cadaverine. “NBD” designates the fluorophore 4-chloro-7-nitrobenzofurazan. The amine donor preferably comprises at least one fluorescent group. The amine donor may comprise at least one dye. In the sense of the present invention, an amine donor is in particularly a molecule which comprises at least one primary amino group on a short aliphatic chain. The primary amino group may, for example, be the ε-amino group of lysine or the free amino group of an unilaterally substituted diamine such as cadaverine or putrescine. Also a substituted carbohydrazide group can be used.

In the reaction scheme 2, for example, an amine donor is shown as R′—NH₂.

In particularly, the amine donor is bound to a glutamine donor substrate by the transglutaminase which is bound to the immobilized glutamine donor. Here, the (thioester) bond between transglutaminase and glutamine donor is broken in the course of an aminolysis. The bond between amine donor and glutamine donor substrate is preferably an amide bond, further preferably a carboxylic acid amide bond.

Preferably, the amine donor is water-soluble. In particularly, the amine donor preferably has a water solubility which is higher than 10 μM. Preferably, the amine donor has a molecular weight in a range of 100 to 1000 Da, for example of 150 to 500 Da. Preferably, the amine donor has a molecular weight of at least 100 Da or at least 150 Da. Preferably, the amine donor has a molecular weight of at most 1000 Da or of at most 500 Da.

The extent of the bond of the amine donor to the glutamine donor substrate is preferably proportional to the activity of the transglutaminase which is bound to the immobilized glutamine donor. The activity of the transglutaminase which is bound to the immobilized glutamine donor in turn is preferably proportional to the portion of the transglutaminase in the sample or to the enzyme activity of the transglutaminase which is present in the sample.

The particular details of the contacting of the transglutaminase which is bound to the immobilized glutamine donor with the amine donor basically depend on the kind of the immobilization of the glutamine donor.

When the immobilized glutamine donor is present as a suspension, in particularly in a reaction vessel, then the contacting may, for example, be realized by the addition of the amine donor to the suspension into the reaction vessel. But on the other hand, when the glutamine donor is present in an immobilized form on a solid-state body, such as for example a test strip, then the contacting can be realized by bringing the amine donor and the solid-state body, in particularly the test strip, into contact. For example, the amine donor can be applied at the site on the solid-state body on which also the glutamine donor is immobilized. However, it is also possible to apply the amine donor at a site on which the glutamine donor is not immobilized. This may be a preferred option in particularly in the case, when the solid-state body, in particularly the test strip, is designed such that the amine donor can migrate through the solid-state body, for example by capillary forces, and thus can be brought into contact with the immobilized glutamine donor.

The amine donor is present and/or during contacting preferably present in solution. Preferred buffer solutions contain approximately isotonic concentrations of NaCl as well as buffer substances such as phosphate or HEPES, in particularly having a pH value in a range of 6.0 to 9.0, preferably in a range of 7.0 to 8.0. pH values which are too low or too high could promote the hydrolysis of the thioester and thus worsen the signal. The pH value is preferably at least 6.0 or at least 7.0. The pH value is preferably at most 9.0 or at most 8.0. The buffer solution may contain one or several biocides, for example NaN₃, preferably in concentrations of 0.01 to 1.0 mM or of 0.05 to 0.5 mM, for example about 0.1 mM.

According to step f) of the method according to the present invention, the enzymatic activity of the transglutaminase is determined with the help of a signal which is created by the bond of the amine donor to the glutamine donor substrate. The signal may in particularly be proportional to the rate of the bond of the amine donor to the glutamine donor substrate which is catalyzed by the transglutaminase. Thus, the signal may be proportional to the temporal increase of the amine donor which is bound to the glutamine donor substrate. In such embodiments the following is true: the higher the enzymatic activity of the transglutaminase in the sample, the higher the rate of the bond of the amine donor to the glutamine donor substrate and, correspondingly, the signal. The signal may in particularly be a spectral change per time unit.

For reducing the background signal, prior to the determination of the amount of the bound amine donor one or several washing steps can be conducted. In particularly, one or several washing steps can be conducted such as described with respect to the removal of constituents of the sample which are not bound to the substrate according to step d). The number of the washing steps is preferably in a range of 1 to 3, or a continuous overflow with washing buffer is used. With the washing steps in particularly amine donor which is bound to the glutamine donor substrate cannot be removed.

Preferably, the signal is determined optically. For example, the signal can be determined with the help of photometry. The signal can in particularly be determined by means of fluorimetry.

The particular details of the determination of the signal in particularly depend on the fluorophore used. Some fluorophores, for example dansyl, change, for example, their absorption maximum during incorporation into the glutamine donor substrate (protein) so that also an intensity ratio emission (incorporated)/emission (not incorporated) or its temporal change can be measured. In particularly in the case of such embodiments it is not necessary to conduct one or several washing steps.

The time from the contacting of the sample with the immobilized glutamine donor to the determination of the enzymatic activity of the transglutaminase is preferably in a range of 1 to 10 minutes, for example of 2 to 5 minutes. The time may, for example, be at least 1 minute or at least 2 minutes. The time may, for example, be at most 5 minutes or at most 10 minutes.

Particularly preferably, the method according to the present invention comprises the following steps:
a) providing of an immobilized FXIII substrate, wherein the FXIII substrate is a peptide, in particularly a polypeptide or protein, and wherein the FXIII substrate comprises at least one glutamine residue,
b) activation of the FXIII which is present in the sample,
c) contacting of the sample with the FXIII substrate so that FXIII which is present in the sample binds to the FXIII substrate,
d) removal of constituents of the sample which are not bound to the FXIII substrate,
e) contacting of the FXIII which is bound to the FXIII substrate with an amine donor so that the amine donor is bound to the FXIII substrate by FXIII, and
f) determination of the enzymatic activity of FXIII with the help of a signal which is created by the bond of the amine donor to the FXIII substrate.

In particularly, the signal may be proportional to the rate of the bond of the amine donor to the FXIII substrate which is catalyzed by FXIII.

The present invention also relates to a device for the determination of the enzymatic activity of transglutaminases, for example of blood coagulation factor XIII, in particularly a test strip, for example in the form of a lateral flow test. The device is preferably a device for conducting the method according to the present invention.

The device comprises at least one reaction zone, preferably exactly one reaction zone.

Preferably, the device comprises three or more zones, in particularly three zones. Preferably, the device comprises at least one application zone, at least one reaction zone and at least one suction zone. The device may comprise a carrier (for example made of a carrier material) onto which the materials of the different zones are applied. The presence of a carrier is an optional feature. In some embodiments of the invention the device does not comprise a carrier.

Preferably, the reaction zone contains an immobilized glutamine donor, in particularly in an amount of 0.01 to 10 mg, for example of 0.05 to 5 mg or of 0.1 to 2 mg. The amount may, for example, be at least 0.01 mg, at least 0.05 mg or at least 0.1 mg. The amount may, for example, be at most 10 mg, at most 5 mg or at most 2 mg.

The immobilized glutamine donor is in particularly a glutamine donor substrate, preferably an FXIII substrate. The immobilized glutamine donor is in particularly a protein. Preferred are proteins having a molecular weight of 10 to 300 kDa and a glutamine content of 3 to 15%. Preferred are proteins having a molecular weight of 10 to 300 kDa, for example of 10 to 100 kDa or of 12 to 30 kDa. The molecular weight of the glutamine donor may, for example, be at least 10 kDa, or at least 12 kDa. The molecular weight of the glutamine donor may, for example, be at most 300 kDa, at most 100 kDa or at most 30 kDa. Preferably, the glutamine content of the glutamine donor is in a range of 3% to 15%, based on the total number of residues, for example in a range of 5% to 12% or of 7% to 10%. The glutamine content of the glutamine donor may, for example, be at least 3%, at least 5% or at least 7%. The glutamine content of the glutamine donor may, for example, be at most 15%, at most 12% or at most 10%.

The immobilization of the glutamine donor preferably comprises one or several of the following measures:
precipitation and adsorption
ionic bond to DEAE-functionalized surfaces
covalent bond by formaldehyde, glutardialdehyde
covalent bond using EDC/NHS in the case of amine- or COOH-functionalized carrier surface
covalent bond by dimethyl suberimidate, dimethyl pimelimidate, dimethyl adipimidate in the case of amine-functionalized carrier surface
covalent bond by carrier-bound epoxides, isocyanates
covalent bond via amino groups to BrCN-activated polysaccharides.

The immobilized glutamine donor may comprise modifications, for example chemical modifications. Particularly preferred are modifications of lysine residues. In particularly, the glutamine donor may be methylated, acetylated or succinylated, for example dimethylated, in particularly N,N-dimethylated or also acetamidinated. Particularly preferably, the glutamine donor is selected from the group consisting of casein and lysozyme, in particularly dimethylcasein and succinylated lysozyme.

The reaction zone is the zone of the device in which the bond of the transglutaminase to the immobilized glutamine donor takes place (in particularly according to step c) of the method) and in which the bond of the amine donor to the glutamine donor substrate takes place (in particularly according to step e) of the method). Preferably, the device comprises exactly one reaction zone. In an alternative, also two or more reaction zones may be provided.

The application zone, the reaction zone and the suction zone may comprise the same materials or may consist of the same materials. But they can also comprise different materials or consist of different materials. Suitable are in particularly all porous materials which allow a capillary-driven transport of liquids and substances which are dissolved or suspended therein. Examples of such materials are, for instance, nitrocellulose membranes, mats of glass fibers, papers, filters made of paper or hydrophilized polymer fibers (e.g., polyester, polypropylene) as well as all composites made of the aforementioned materials. Here, the materials can, for example, consist of woven or unwoven fibers or threads, but also of open or closed foams.

Preferably, the device comprises or consists of the following materials: unwoven mats made of paper, polymer or glass fibers.

The device may comprise an enclosure, for example an enclosure made of plastic.

In addition to the materials of the application zone, the reaction zone and the suction zone, the device may comprise one or several further materials, for example a carrier material. The carrier material may, for example, consist of glass, plexiglass, a polymer film or also of a coating which is a barrier for the liquid which is used in the device. However, such a carrier material is not mandatory, since air surrounding the device is already a sufficient (and hydrophobic) barrier.

Preferably, the application zone comprises or consists of the following materials: unwoven mats made of paper, polymer or glass fibers

Preferably, the reaction zone comprises or consists of the following materials: unwoven mats made of paper, polymer or glass fibers

Preferably, the suction zone comprises or consists of the following materials: paper or fiber mat having an absorption capacity which is higher than that of the application and reaction zones. This can be achieved by a higher thickness, length or also width of the fiber material of the suction zone. The decisive factor is the absorption capacity, not the geometry.

Preferably, the thickness or length of the suction zone is at least twice as great as the thickness or length of the application zone or as the thickness or length of the reaction zone. Preferably, the application zone and the reaction zone have the same or substantially the same thickness. Preferably, the thickness of the suction zone is in a range from 2 times the thickness of the application zone or the reaction zone up to 10 times the thickness of the application zone or the reaction zone.

Preferably, the absorption capacity of the suction zone is at least twice as great as the absorption capacity of the application zone or as the absorption capacity of the reaction zone. Preferably, the application zone and the reaction zone have the same or substantially the same absorption capacity. Preferably, the absorption capacity of the suction zone is in a range from 2 times the absorption capacity of the application zone or the reaction zone up to 10 times the absorption capacity of the application zone or the reaction zone.

The device may contain the application zone and the reaction zone in one combined application/reaction zone. However, preferably, the application zone and the reaction zone are located at spatially different positions of the device. However, this does not mean that there is no connection between the application zone and the reaction zone. On the contrary, the device comprises at least one connection, preferably exactly one connection between the application zone and the reaction zone.

The application zone is the zone of the device in which preferably the sample and/or the amine donor and/or the washing solution is applied. It is possible to provide different application zones, for example at least one application zone for applying the sample, at least one application zone for applying the amine donor and/or at least one application zone for applying the washing solution. Preferably, however, the application zone of the device is a combined application zone, in which the sample, the amine donor and the washing solution can be applied. Preferably, the device contains exactly one application zone.

The connection of the application zone and the reaction zone makes it possible that a sample which is applied in the application zone and/or an amine donor which is applied in the application zone and/or a washing solution which is applied in the application zone can get from the application zone to the reaction zone. The transport from the application zone into the reaction zone may in particularly occur by means of capillary forces.

Preferably, the device contains exactly one suction zone. In an alternative, also two or more suction zones may be provided. Preferably, the device contains at least one connection, preferably exactly one connection between the reaction zone and the suction zone. The suction zone in particularly is useful to remove reagents which are not bound to the immobilized glutamine donor, such as for example unbound amine donor, and sample constituents which are not bound to the immobilized glutamine donor (in particularly blood constituents) from the reaction zone. Also an excess of the washing solution or other solutions can be removed from the reaction zone with the help of the suction zone. The transport from the reaction zone into the suction zone can take place in particularly by means of capillary forces.

The device preferably contains exactly two connections, namely one connection between the application zone and the reaction zone as well as one connection between the reaction zone and the suction zone. The connections are capillary-active so that an uninterrupted liquid transport is guaranteed. When the zones are manufactured from different materials, then the connection between the zones can consist of the material of one of both zones or of a mixture of the materials or it can comprise them. It is also possible that the material of the connection comprises or consists of a further material which is not comprised in one of both connected zones. When the zones are designed such that there is no change of material, then the connections are preferably made of the same material. For example, the entire device can also be manufactured of a fiber mat, e.g., paper.

### EXAMPLES

Material and Methods for all Examples (if Applicable)

Instruments
UV lamp 365 nm: neolab
Camera: P 40 Lite (Huawei)
Imager (inter alia for an excitation at 460 nm): Fusion FX 7 Edge (Vilber)

Buffers and Papers
Standard buffer: 50 mM HEPES, 100 mM NaCl, 0.1 mM NaN₃, pH value 7.4
Reaction solution for the DansCad staining of DEAE-cellulose/dimethylcasein: standard buffer with dansylcadaverine (50 μM), DTT (2 mM) and CaCl₂ (5 mM)
Reaction solution for LFT (lateral flow test): like in the case of cellulose staining, but 350 μM dansylcadaverine
Paper for application and reaction zone: Whatman 1 CHR (chromatography)
Suction paper: Whatman gel blot paper

Enzymes

FXIII: Fibrogammin (CSL Behring). The amount is given in International Units [I.U.]. These units are determined by the producer through a comparison with a standardized normal plasma.

### Example 1 (Bond of FXIII to Dimethylcasein Adsorbed on DEAE-Cellulose)

0.625 I.U. of FXIIIₐ were incubated for 10 min with an immobilized glutamine donor substrate (1.25 mg dimethylcasein on 125 mg DEAE-cellulose). Then, it was washed 0 to 4 times with standard buffer, pH value 7.4. Subsequently, 50 μM dansylcadaverine were added as amine donor. Under UV lighting (365 nm) the fluorescence of the dansylcadaverine bound to dimethylcasein was detected.

Surprisingly it has been found that FXIIIₐ binds to the immobilized glutamine donor protein sufficiently strongly so that it can be extracted and enriched from a sample.

Washing once or twice does not visibly reduce the fluorescence. Thus, a large part of the FXIII added is immobilized. Also in the case of several washing steps, FXIII can be detected very well.

### Example 2 (Reversible Bond of FXIII to Dimethylcasein-DEAE-Cellulose)

In comparison to example 1, only the washing steps were altered.

Example 2A: reaction with dansylcadaverine after three washing steps with standard buffer

Example 2B: reaction after two washing steps with 0.1 M glycine ethyl ester for the aminolysis of the intermediate, one washing step with buffer

Example 2C: two washing steps with 0.1 M glycine ethyl ester+10 μM silver nitrate for blocking reactive thiols, one washing step with buffer

It was possible to break the bond of FXIII to dimethylcasein with amine donors. A blocking of the essential —SH group with Ag⁺ ions was not necessary; in the presence of the amine donor FXIII remained in solution. Both, the bond and also the staining, are based on the specific activity of the analyte FXIII.

### Example 3 (Detection of FXIII on the LFT in the Dansylcadaverine Test)

The Examples 1 and 2 were conducted in reaction vessels (Eppendorf tubes). In contrast thereto, the Example 3 was conducted with a device having an application zone, a reaction zone and a suction zone. For the detection of FXIII a paper-based, very simple manually manufactured lateral flow test (LFT) was suitable.

The analyte, rinsing and reaction solutions were applied onto the application zone one after the other. 50 μl of analyte were sucked up within a time of ca. 20 seconds; then, 100 μL of washing buffer followed for transporting the analyte into the reaction zone. After an incubation for 10 minutes for bonding FXIII to the substrate 100 μL of reaction solution followed for washing out unbound components.
Analyte: 0/0.313/0.625/1.25 I.U. of FXIII in standard buffer with 200 mM CaCl₂.
Washing buffer: standard buffer with 200 mM CaCl₂
Reaction solution: 350 μM dansylcadaverine, 2 mM dithiothreitol in standard puffer

The increase of the fluorescence was recorded in the imager at room temperature. Between 5 and 30 minutes the dependence shown in the figure was revealed. The results are shown in FIG. 2.

Since it is basically a linear increase of the intensity, also measurements after shorter times are possible.

When instead of the FXIII solution a strongly fluorescent mixture of serum-albumin (50 mg/ml) and dansylcadaverine (350 μM) in standard buffer was used, then after washing with standard buffer inclusive 50 μM dansylcadaverine in the reaction zone only the weak fluorescence of the unbound dansylcadaverine was observed. The strong fluorescence of the albumin-bound dansylcadaverine was visible in the suction zone. Thus, the protein has been washed out from the reaction zone. Thus, the FXIII-specific reaction shown in FIG. 2 is based on the bond and retention of the analyte FXIII to (or on) the Ca²⁺-precipitated dimethylcasein.

### Example 4 (Reaction in the Presence of Potential Interfering Components)

The test structure and the conditions were the same like in Example 3.

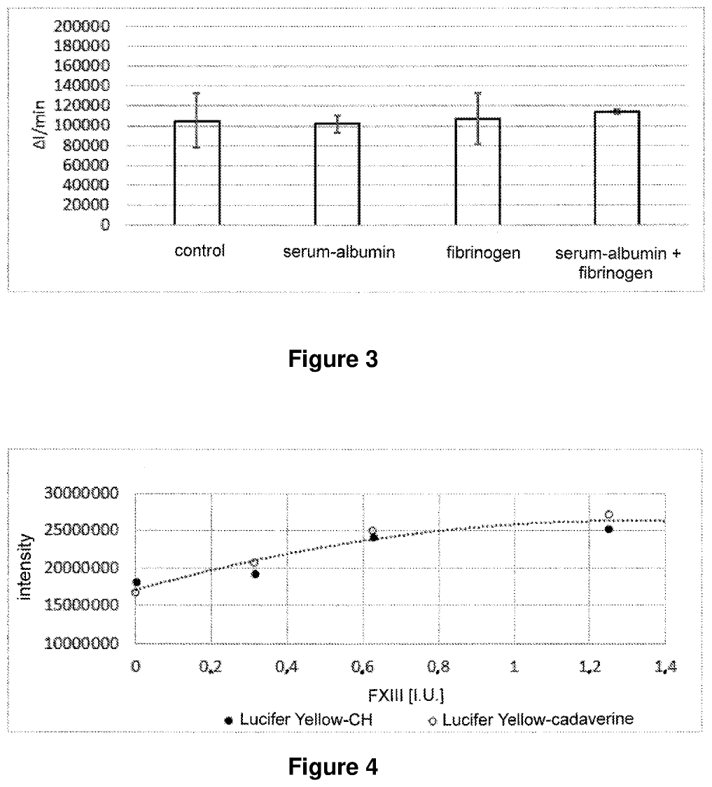

The bond of FXIII and the staining with dansylcadaverine were independent of added fibrinogen (5 mg/ml) or serum-albumin (50 mg/ml) also in the high physiologically present concentrations (see FIG. 3). The preference of the non-physiological substrate was surprising. Here, a removal of fibrinogen is absolutely not necessary.

### Example 5 (FXIII Dependence, when Lucifer Yellow is Incorporated)

It is known that also other fluorophores in dependence on the polarity of the environment change their fluorescence intensity, e.g., dapoxyl, aminonaphthalimide or NBD fluorophores. Since the reaction with FXIII independently of the chromophore can successfully be conducted with many amines, by the selection of the fluorophore the wavelengths for excitation and emission can be chosen appropriately.

Also without this change of the fluorescence FXIII can be detected. Suitable are, e.g., Lucifer Yellow-CH and Lucifer Yellow-cadaverine. The reaction was conducted in the lateral flow structure analogously to the method with dansylcadaverine (see example 3). Since, however, the fluorescence of the dye Lucifer Yellow does not substantially change, when it is incorporated, after a duration of exposure of 20 minutes it was washed twice with standard buffer+200 mM CaCl₂, and subsequently the fluorescence was measured with the imager. The results are shown in FIG. 4.

Thus, the detection works independently of the chromophore. Also the carbohydrazide in the Lucifer Yellow-CH is accepted as amine donor, similarly like in the case of the cadaverine group. In comparison to dansylcadaverine, in the case of Lucifer Yellow the longer excitation wavelength is advantageous. But due to the lack of polarity sensitivity additional washing steps are required for the removal of the unreacted dye. Measurements by means of remission photometry are also possible. When required, dyes with high absorbance coefficients or even cadaverine-modified nanogold particles can be used for this purpose.

### LIST OF REFERENCE SIGNS

11 application zone
12 reaction zone
13 suction zone
14 carrier

What is claimed is:

1. A method for the determination of the enzymatic activity of a transglutaminase in a sample, wherein the method comprises the following steps:
a) providing of an immobilized glutamine donor, wherein the glutamine donor is a peptide, in particularly a polypeptide or protein, and wherein the glutamine donor comprises at least one glutamine residue,
b) activation of the transglutaminase which is present in the sample,
c) contacting of the sample with the glutamine donor so that transglutaminase which is present in the sample binds to the glutamine donor,
d) removal of constituents of the sample which are not bound to the glutamine donor,
e) contacting of the transglutaminase which is bound to the glutamine donor with an amine donor so that the amine donor is bound to a glutamine donor substrate by the transglutaminase, and
f) determination of the enzymatic activity of the transglutaminase with the help of a signal which is created by the bond of the amine donor to the glutamine donor substrate.

2. The method according to claim 1, wherein the sample is a blood sample.

3. The method according to claim 1, wherein the glutamine donor is bound to a carrier.

4. The method according to claim 1, wherein step c) comprises an incubation step and wherein the incubation time is in a range of 10 seconds to 5 minutes.

5. The method according to claim 1, wherein the glutamine donor is the glutamine donor substrate.

6. The method according to claim 1, wherein the bond between transglutaminase and glutamine donor is a covalent bond between an amino acid in the active center of the glutaminase and a glutamine residue of the glutamine donor.

7. The method according to claim 1, wherein the bond between transglutaminase and immobilized glutamine donor is a thioester bond.

8. The method according to claim 1, wherein step d) comprises one or several washing steps.

9. The method according to claim 1, wherein the amine donor comprises at least one fluorescent group or at least one dye.

10. The method according to claim 1, wherein the transglutaminase is blood coagulation factor XIII.

11. The method according to claim 1, wherein the signal is determined optically.

12. The method according to claim 1, wherein the amine donor is dansylcadaverine, dapoxylcadaverine or NBD-cadaverine.

13. A device, in particularly for conducting the method according to claim 1, for the determination of the enzymatic activity of a transglutaminase in a sample, wherein the device comprises at least one reaction zone and wherein the reaction zone comprises immobilized glutamine donor.

14. The device according to claim 13, wherein the glutamine donor is a protein having a molecular weight of 10 to 300 kDa.

15. The device according to claim 13, wherein the device comprises at least one application zone and wherein the device is designed such that by means of capillary forces sample which is applied in the application zone can reach the reaction zone.

16. The device according to claim 13, wherein the immobilized glutamine donor is present in an amount from 0.01 to 10 mg.
